(19) United States
(12) Patent Application Publication
(10) Pub. No.: US 2014/0276050 A1
(43) Pub. Date: Sep. 18, 2014

(54) ULTRASOUND RENAL NERVE ABLATION AND IMAGING CATHETER WITH DUAL-FUNCTION TRANSDUCERS

(71) Applicant: BOSTON SCIENTIFIC SCIMED, INC., Maple Grove, MN (US)

(72) Inventors: MARK L. JENSON, GREENFIELD, MN (US); ROGER N. HASTINGS, MAPLE GROVE, MN (US); SCOTT R. SMITH, CHASKA, MN (US)

(73) Assignee: BOSTON SCIENTIFIC SCIMED, INC., MAPLE GROVE, MN (US)

(21) Appl. No.: 14/194,371

(22) Filed: Feb. 28, 2014

Related U.S. Application Data
(60) Provisional application No. 61/780,859, filed on Mar. 13, 2013.

(51) Int. Cl. A61B 8/00; A61B 8/12; A61B 8/08; A61B 17/32

(52) U.S. Cl. CPC A61B 8/445; A61B 17/320016; A61B 17/320068; A61B 8/12; A61B 8/4483; A61B 8/56; A61B 8/08
USPC 600/439

(57) ABSTRACT

Systems for nerve and tissue modulation are disclosed. An example system may include an intravascular nerve modulation system including an elongated shaft having a proximal end region and a distal end region. The system may further include one or more dual-function ultrasound transducers for performing imaging and tissue modulation.

### CROSS-REFERENCE TO RELATED APPLICATIONS

This application claims priority under 35 U.S.C. §119 to U.S. Provisional Application Ser. No. 61/780,859, filed Mar. 13, 2013, the entirety of which is incorporated herein by reference.

### TECHNICAL FIELD

The present disclosure relates generally to methods and apparatuses for nerve modulation techniques such as ablation of nerve tissue or other modulation techniques through the walls of blood vessels.

### BACKGROUND

Certain treatments may require the temporary or permanent interruption or modification of select nerve function. One example treatment is renal nerve ablation, which is sometimes used to treat conditions related to congestive heart failure or hypertension. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves (and nervous tissue such as brain tissue), including renal nerves, run along the walls of or in close proximity to blood vessels and thus can be accessed intravascularly through the walls of the blood vessels. In some instances, it may be desirable to ablate perivascular nerves using ultrasonic energy. In other instances, the perivascular nerves may be ablated by other means including application of thermal, radiofrequency, laser, microwave, and other related energy sources to the target region. Combination ultrasound devices with both ablation and imaging transducers may require more complicated catheters and power units. This may result in a catheter that is stiffer than desired. Furthermore, the apparatus may be more costly to manufacture. It may be desirable to provide for alternative systems and methods for intravascular nerve modulation that provide both imaging acoustics and ablation acoustics.

### SUMMARY

This disclosure is directed to several alternative designs, materials and methods of manufacturing medical device structures and assemblies for performing nerve ablation.

Accordingly, one illustrative embodiment is a system for intravascular nerve modulation system that may include an elongate shaft having a proximal end region and a distal end region. A first and a second transducer array may be disposed adjacent to the distal end region of the elongate shaft. The first and second transducer arrays include dual-function transducers configured to provide both imaging acoustics and modulation acoustics. The nerve modulation may include a first mode where at least one of the first or second transducer arrays is configured to send ultrasonic pulses to image a target region and the other of the first or second transducer arrays is configured to receive reflected pulses and a second mode where both the first and second transducer arrays are configured to send ultrasonic pulses to modulate the target region.

Another illustrative embodiment is a method for performing intravascular nerve modulation. A nerve modulation system including an elongate shaft having a proximal end region and a distal end region may be provided. The modulation system may further include a first transducer array having one or more transducers disposed adjacent to the distal end region of the elongate shaft and a second transducer array having one or more transducers disposed adjacent to the distal end region of the elongate shaft. The nerve modulation system may be advanced through a lumen such that the distal end region of the elongate shaft is adjacent to a first target region. A first current may be supplied to one of the first or second transducer arrays to generate a first acoustic energy. Reflected pulses from the first acoustic energy may be received at one of the first or second transducer arrays to image the first target region. A second current may be supplied to both the first and the second transducer arrays to generate a second acoustic energy different from the first acoustic energy.

The above summary of an example embodiment is not intended to describe each disclosed embodiment or every implementation of the present disclosure.

### BRIEF DESCRIPTION OF THE DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 illustrates a portion of an example intravascular nerve modulation system.

FIG. 3-8 illustrate portions of an example modulation procedure.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may be indicative as including numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

Although some suitable dimensions ranges and/or values pertaining to various components, features and/or specifications are disclosed, one of the skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

For purposes of this disclosure, “proximal” refers to the end closer to the device operator during use, and “distal” refers to the end further from the device operator during use.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with one embodiment, it should be understood that such feature, structure, or characteristic may also be used connection with other embodiments whether or not explicitly described unless cleared stated to the contrary.

Certain treatments require the temporary or permanent interruption or modification of select nerve function. One example treatment is renal nerve ablation, which is sometimes used to treat conditions related to congestive heart failure or hypertension. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

While the devices and methods described herein are discussed relative to renal nerve modulation, it is contemplated that the devices and methods may be used in other locations and/or applications where nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, tumor ablation, benign prostatic hyperplasia therapy, nerve excitation or blocking or ablation, modulation of muscle activity, hyperthermia or other warming of tissues, etc. In some instances, it may be desirable to ablate perivascular renal nerves with ultrasound ablation.

Ultrasound energy may be used to generate heat at a target location. The high frequency sound waves produced by an ultrasonic transducer may be directed at a target region and absorbed at the target region. As the energy emitted is absorbed, the temperature of the target region may rise. In order to perform renal nerve ablation, target nerves must be heated sufficiently to make them nonfunctional, while thermal injury to the artery wall is undesirable. In some instances, catheter-based ultrasound devices may be used to monitor the target region for changes due to ablation and/or to image the target tissue. Ultrasound imaging catheters can incorporate a single transducer or an array of transducers typically tuned to a higher frequency, such as, but not limited to, about 40 megahertz (MHz). A conventional imaging approach may use the same transducers for both sending ultrasonic pulses and for receiving the reflected pulses for imaging. These transducers typically have a backing layer to reduce “ringing” which would degrade the image, but which can also reduce the acoustic output (and reflected pulses). Catheter-based ultrasound devices may also be used to ablate target tissue a short distance away from the catheter. Ultrasound ablation catheters can incorporate a single transducer or an array of transducers, typically tuned to a lower frequency such as, but not limited to, about 10 MHz, with no backing layer, which would reduce energy output. A multiple-transducer array may be used for preferentially ablating at a desired location, and the frequencies may be chosen depending on the depth and nature of target tissue.

The optimal design of imaging and ablation transducers is different due to differences in transducer damping, tissue depth and attenuation, and optimal frequency. Thus, combination ultrasound devices with both ablation and imaging transducers may require more complicated catheters and power units than devices including only ablation or imaging transducers. Dual-function transducers may provide both imaging acoustics and ablation acoustics in a more efficient device to reduce the number and/or size of the transducers.

FIG. 1 is a schematic view of an illustrative renal nerve modulation system 10 in situ. System 10 may include an element 12 for providing power to a transducer disposed adjacent to, about, and/or within a central elongated shaft 14 and, optionally, within a guide catheter 16. A proximal end of element 12 may be connected to a control and power element 18, which may supply the necessary electrical energy to activate the one or more transducers at or near a distal end of the element 12. The control and power element 18 may include monitoring elements to monitor parameters such as power, temperature, voltage, pulse size and/or frequency and other suitable parameters as well as suitable controls for performing the desired procedure. In some instances, the power element 18 may control an ultrasound ablation transducer. The ablation transducer may be configured to operate at a frequency of about 9-10 megahertz (MHz). It is contemplated that any desired frequency may be used, for example, from 1-20 MHz. In addition, it is contemplated that frequencies outside this range may also be used, as desired. While the term “ultrasound” is used herein, this is not meant to limit the range of vibration frequencies contemplated. For example, it is contemplated that the perivascular nerves may be ablated by other means including application of thermal, radiofrequency, laser, microwave, and other related energy sources to the target region.

FIG. 2 is an illustrative embodiment of a distal end of a renal nerve modulation system 100 disposed within a body lumen 102 having a vessel wall 104. The vessel wall 104 may be surrounded by local body tissue. The local body tissue may comprise adventitia and connective tissues, nerves, fat, fluid, etc. in addition to the muscular vessel wall 104. The system 100 may include an elongate shaft 106 having a distal end region 108. The elongate shaft 106 may extend proximally from the distal end region 108 to a proximal end configured to remain outside of a patient's body. The proximal end of the elongate shaft 106 may include a hub attached thereto for connecting other treatment devices or providing a port for facilitating other treatments. It is contemplated that the stiffness of the elongate shaft 106 may be modified to form a modulation system 100 for use in various vessel diameters and various locations within the vascular tree. The elongate shaft 106 may further include one or more lumens extending therethrough. For example, the elongate shaft 106 may include a guidewire lumen and/or one or more auxiliary lumens. The lumens may be configured in any way known in the art. For example, the guidewire lumen may extend the entire length of the elongate shaft 106 such as in an over-the-wire catheter or may extend only along a distal portion of the elongate shaft 106 such as in a single operator exchange (SOE) catheter. These examples are not intended to be limiting, but rather examples of some possible configurations. While not explicitly shown, the modulation system 100 may further include temperature sensors/wire, an infusion lumen, radiopaque marker bands, fixed guidewire tip, a guidewire lumen, external sheath, centering basket, and/or other components to facilitate the use and advancement of the system 100 within the vasculature.

The system 100 may include a first array 110 of ultrasound transducers including one or more transducers 112a, 112b, 112c, 112d (collectively 112) and a second array 115 of ultrasound transducers including one or more transducers 114a, 114b, 114c, 114d (collectively 114) positioned adjacent the distal end region 108 of the elongate shaft 106. However, the transducer arrays 110, 115 may be placed at any longitudinal location along the elongate shaft 106 desired. In some instances, the modulation system 100 may include more than two transducer arrays 110, 115. It is contemplated that while each array 110, 115 is illustrated as including four transducers 112a, 112b, 112c, 112d, 114a, 114b, 114c, 114d, each array 110, 115 may include any number of transducers desired, such as, but not limited to one, two, three, four, five, or more. In some instances, the first transducer array 110 may be staggered with the second transducer array 115 such that the transducers 112 in the first array 110 are not positioned adjacent to one another and the transducers 114 in the second array 115 are not positioned adjacent to one another. In other words, the transducers 112 in the first array 110 and the transducers 114 in the second array 115 may alternate. However, this is not required. In some instances, two or more of the transducers 112 in the first array 110 may be positioned adjacent to one another and two or more of the transducers 114 in the second array 115 may be positioned adjacent to one another. It is contemplated that the transducers 112, 114 may be positioned in any orientation desired. While the Figures illustrate the transducers 112, 114 as oriented in a line, or along a longitudinal axis, other orientations or patterns are contemplated. For example, in some embodiments, some transducers may be oriented towards a first side of the vessel wall while other transducers may be oriented to face another portion of the wall, such as but not limited to, generally opposite the first side. It is contemplated that the transducers 112, 114 may be arranged in other patterns as well, such as, but not limited to, a ring pattern or a helical pattern. These are just examples.

It is further contemplated that more than one row of transducers 112, 114 may be disposed on the elongate shaft 106. In some instances, the transducer arrays 110, 115 may include a number of transducers (two, three, four, or more) spaced about the circumference of the elongate shaft 106. This may allow for ablation of multiple circumferential locations about the body lumen simultaneously. In other embodiments, the transducer arrays 110, 115 may comprise a focused or phased array of transducers. In such a configuration, the arrays 110, 115 may be configured to be directed at a focus region such that multiple transducers are radiating energy at a common target region. The transducer arrays 110, 115 may be configured such that the timing of activations can be offset or “phased” in order to preferentially focus the applied acoustic energy at a target area for both imaging and ablation purposes. It is contemplated that a sequence of varying focal points can be targeted by varying the timing of the phased array.

The transducers 112, 114 may be formed from any suitable material such as, but not limited to, lead zirconate titanate (PZT). It is contemplated that other ceramic or piezoelectric materials may also be used. While not explicitly shown, the transducers 112, 114 may have a first radiating surface, a second radiating surface, and a perimeter surface extending around the outer edge of the transducers 112, 114. In some instances, the transducers 112, 114 may include a layer of gold, or other conductive layer, disposed on the first and/or second side over the PZT crystal for connecting electrical leads to the transducers 112, 114. In some embodiments, the transducers 112, 114 may be structured to radiate acoustic energy from a single radiating surface. In such an instance, one radiating surface may include a backing layer to direct the acoustic energy in a single direction. In other embodiments, the transducers 112, 114 may be structured to radiate acoustic energy from two radiating surfaces. In some instances, one or more tie layers may be used to bond the gold to the PZT. For example, a layer of chrome may be disposed between the PZT and the gold to improve adhesion. In other instances, the transducers 112, 114 may include a layer of chrome over the PZT followed by a layer of nickel, and finally a layer of gold. These are just examples. It is contemplated that the layers may be deposited on the PZT using sputter coating, although other deposition techniques may be used as desired. While the transducers 112, 114 are described as ultrasonic transducers, it is contemplated that other methods and devices for raising the temperature of the nerves may be used, such as, but not limited to: radiofrequency, microwave, or other acoustic, optical, electrical current, direct contact heating, or other heating.

It is contemplated that the radiating surface (surface which radiates acoustic energy) of the transducers 112, 114 may take any shape desired, such as, but not limited to, square, rectangular, polygonal, circular, oblong, etc. In some embodiments, the transducers 112, 114 may be cylindrical and extend around the circumference of the elongate shaft 106. The acoustic energy from the radiating surface of the transducers 112, 114 may be transmitted in a spatial pressure distribution related to the shape of the transducers 112, 114. With exposures of appropriate power and duration, lesions formed during ablation may take a shape similar to the contours of the pressure distribution. As used herein, a “lesion” may be a change in tissue structure or function due to injury (e.g. tissue damage caused by the ultrasound). Thus, the shape and dimensions of the transducers 112, 114 may be selected based on the desired treatment and the shape best suited for that treatment. It is contemplated that the transducers 112, 114 may also be sized according to the desired treatment region. For example, in renal applications, the transducers 112, 114 may be sized to be compatible with a 6 French guide catheter, although this is not required.

In some embodiments, the transducers 112, 114 may be formed of a separate structure and attached to the elongate shaft 106. For example, the transducers 112, 114 may be bonded or otherwise attached to the elongate shaft 106. In some instances, the transducers 112, 114 may include a ring or other retaining or holding mechanism (not explicitly shown) disposed around the perimeter of the transducers 112, 114 to facilitate attachment of the transducers 112, 114 to the elongate shaft 106. The transducers 112, 114 may further include a post, or other like mechanism, affixed to the ring such that the post may be attached to the elongate shaft 106 or other member. In some instances, the rings may be attached to the transducers 112, 114 with a flexible adhesive, such as, but not limited to, silicone. However, it is contemplated that the rings may be attached to the transducers 112, 114 in any manner desired. While not explicitly shown, in some instances, the elongate shaft 106 may be formed with grooves or recesses in an outer surface thereof. The recesses may be sized and shaped to receive the transducers 112, 114. For example, the transducers 112, 114 may be disposed within the recess such that a first surface contacts the outer surface of the elongate shaft 106 and a second surface is directed towards a desired treatment region. However, it is contemplated that the transducers 112, 114 may be affixed to the elongate shaft in any manner desired.

In some embodiments, the transducers 112, 114 may be affixed to an outer surface of the elongate shaft 106 such that the surface(s) of the transducers 112, 114 are exposed to blood flow through the vessel. As the power is relayed to the transducers 112, 114, the power that does not go into generating acoustic power generates heat. As the transducers 112, 114 heat, they become less efficient, thus generating more heat. Passive cooling provided by the flow of blood may help improve the efficiency of the transducers 112, 114. However, in some instances, additional cooling may be provided by introducing a cooling fluid or other cooling mechanism to the modulation system 100.

While not explicitly shown, the transducers 112, 114 may be connected to a control unit (such as control unit 18 in FIG. 1) by electrical conductor(s). In some embodiments, the electrical conductor(s) may be disposed within a lumen of the elongate shaft 106. In other embodiments, the electrical conductor(s) may extend along an outside surface of the elongate shaft 106. The electrical conductor(s) may provide electricity to the transducers 112, 114 which may then be converted into acoustic energy. The acoustic energy may be directed from the transducers 112, 114 in a direction generally perpendicular to the radiating surfaces of the transducers 112, 114, as illustrated at lines 116, 118. As discussed above, acoustic energy radiates from the transducers 112, 114 in a pattern related to the shape of the transducers 112, 114 and lesions formed during ablation take shape similar to contours of the pressure distribution.

It is contemplated that each transducer array 110, 115 may be comprised of dual-function transducers. For example, the transducers 112, 114 in the arrays 110, 115 may provide both provide both imaging (detection) acoustics and ablation acoustics. In some embodiments, it is contemplated that each of the transducers 112, 114 may be tuned to a particular frequency, such as, but not limited to 20 MHz, for both imaging and ablation. Alternatively, it is contemplated that the transducers 112, 114 may be tuned for a frequency of approximately 5-10 MHz for ablation, but operated briefly (e.g. for a few microseconds) at an imaging frequency (approximately 20-40 MHz). These are just examples.

In some embodiments, a first set of transducers, for example, but not limited to, array 110, may be tuned for a first frequency, for example, for performing ablation, while a second set of transducers, for example, but not limited to, array 115, may be tuned for a second frequency, for example, for imaging. In this instance, the ablation transducers may be optimized for acoustic output, with little damping, while the imaging transducers may be optimized for receiving. The imaging transducers may be tuned to have a broad bandwidth or to operate over a larger range of frequencies, which may be achieved by damping. This may enable the delivery and detection of short pulses which may be only a few wavelengths in during. This may in turn provide for improved radial (R in the R, Z, theta cylindrical coordinate system) resolution. It is further contemplated that the ablation transducers may be tuned to have a narrow bandwidth or to operate over a smaller range of frequencies, at the expense of damping. This may increase the minimum pulse width, which may increase acoustic energy thus enabling the delivery of more acoustic power with reduced transducer heating. The optimization of the transducers for imaging and ablation may allow the transducers 112, 114 to function more efficiently, thus reducing the number or size of transducers necessary. The second transducer array 115 may receive the initial reflections for imaging from the first transducer array 110 despite being tuned to different frequencies.

The receiving sensitivity of the transducers 112, 114 may be taken into consideration when tuning the transducers. For example, the receiving sensitivity of a transducer may be significantly reduced at frequencies other than resonant frequency thus reducing the signal to noise ratio. Similarly, the efficiency of the transmission transducer may be lower which may lead to heating of the transducer. Sensitivity and efficiency may be somewhat higher at harmonics (multiples) of the resonant frequency, but not as high as at resonant frequency. It is contemplated that, when so provided, dual-function transducers may be tuned such that the imaging frequency is a harmonic of the ablation. It is further contemplated that tissue harmonic imaging may be employed. In this instance, the transducer may be configured to detect reflections from the tissue at multiples of the transmitted frequency.

In some instances, damping may be achieved through a number of different methods. As described above, in some embodiments, a backing layer may be used to provide mechanical damping. In other embodiments, active damping may be achieved through shaped waveform pulses delivered to the transducer. In yet other embodiments, passive damping may be achieved by adding frequency dependent impedances (loads) to the transducer circuit. These loads may be switched by, for example, a DC biased PIN diode. These are just examples.

It is contemplated that transducer arrays 110, 115 may be operated in a number of different combinations. For example, in some instances, both the first array 110 and the second array 115 may be used for both imaging and ablation. In other instances, it is contemplated that only the first transducer array 110 or the second transducer array 115 will be used for imaging while both the first transducer array 110 and the second transducer array 115 are used for ablation. In other embodiments, it is contemplated that only the first transducer array 110 or the second transducer array 115 will be used for imaging while the array that is not used for imaging is used for ablation.

FIG. 2 illustrates an embodiment in which the second transducer array 115 is used for imaging. The first transducer array 110 may be energized in a first imaging mode for imaging. The first transducer array 110 may be energized and acoustic energy 116 may be emitted from each of the transducers 112a, 112b, 112c, 112d in the first array 110. The second transducer array 115 may detect the initial reflections 120 to image or detect tissue changes. Imaging (detection of reflections) may not require the transducer to be energized, except with regard to active damping of the transmitted pulse described above. The second transducer array 115 may only use the initial reflections 120 during imaging to avoid “ringing” or to avoid higher power reflections interfering with the imaging. In some instances, the initial reflection (after internal reflections have dissipated) could be detected with good resolution and would correspond to the internal elastic lamina (inner wall) of the vessel. This may be enhanced if the leading edge of the pulse contains increased high frequency content.

It is contemplated that the transducers 112, 114 may be dual-function transducers such that regardless of which array 110, 115 is used for imaging, all of the transducers 112, 114 may be used for ablation. Once the target region has been imaged, both the first transducer array 110 and the second transducer array 115 may be energized in a second ablation mode such that all of the transducers 112, 114 may emit acoustic energy 116, 118 towards the target region for tissue modulation. After a ring-down period, the cycle may be repeated. The ring down period may allow time for the vibration of the transducers 112,114 to subside. The energy may be dissipated internally and transmitted externally to the surrounding media. This is the transmit pulse width described above. The pulse width may also affect resolution as described by the point spread function. The ring-down period may also allow internal catheter reflections to subside. In some instances, active or passing damping may be employed to shorten the ring-down period and improve resolution without degrading the ablation performance.

It is contemplated that the modulation system 100 may alternate between using the first transducer array 110 or the second transducer array 115 to detect the reflections from the target region, or the first transducer array 110 or the second transducer array 115 may be used exclusively. While FIG. 2 illustrates acoustic energy 116, 118 and the reflections 120 in the same figure, as will be discussed in more detail with respect to FIGS. 3-8, the imaging and ablation cycles may be an iterative process and in some instances may not be accomplished simultaneously. It is contemplated that the progress of the ablation procedure may be monitored periodically using the imaging ability of the transducers 112, 114. The ability of all of the transducers 112, 114 to contribute to ablation while some or all also provide imaging capabilities may maximize acoustic output with fewer transducers relative to other dual function devices.

FIGS. 3-8 illustrate a step by step example modulation procedure using acoustic energy for both imaging and ablation. Turning first to FIG. 3, prior to performing the actual tissue modulation, the target region may be evaluated and/or imaged. This may help determine the types of tissues present in addition to the target tissue (e.g. in the case of renal nerve ablation, nerves) which may help determine the length and/or intensity acoustic energy is applied in an ablation mode. As shown, the first transducer array 110 may be energized in a first imaging mode for imaging. The first transducer array 110 may be energized and acoustic energy 116 may be emitted from each of the transducers 112. The second transducer array 115 may detect or listen to the initial reflections or leading edge of the return signal 120 to image or evaluate the target region and/or tissue changes. It is contemplated the imaging process may last for approximately 10 microseconds. While the first transducer array 110 is described as emitting the acoustic energy and the second transducer array 115 as receiving the reflections, it is contemplated that the reverse configuration may also be used. For example, in some instances, the second transducer array 115 may emit acoustic energy while the first transducer array 110 receives the reflections.

Once the target region has been evaluated, both the first transducer array 110 and the second transducer array 115 may be energized in the second ablation mode, as shown in FIG. 4. Acoustic energy 116, 118 may be directed from both the first transducer array 110 and the second transducer array 115. In the ablation mode, acoustic energy 116, 118 may be directed from the transducers 112, 114 to form lesions in the desired target region. Acoustic energy 116, 118 may be directed towards the target region until another evaluation or image of the target region is desired. Energy delivery to the transducers 112, 114 may be stopped for a ring-down period as shown in FIG. 5. The ring-down period may be a short time period relative the length of time the transducers 112, 114 are activated in the ablation mode. It is contemplated the ring-down period may be approximately 50 microseconds. However, the ring-down period may be any length of time necessary to allow the vibration of the transducers 112, 114 to subside. After the ring-down period, the imaging and ablation cycles may be repeated.

After the ring-down period, the target region may be evaluated and/or imaged to determine if further tissue modulation is necessary and/or how much further tissue modulation is needed, as shown in FIG. 6. This may help determine the length and/or intensity acoustic energy is applied in an ablation mode. It is contemplated that the evaluation of the target region may not require high radial resolution as regional changes in total reflected power may be indicative of lesion formation. As shown, the second transducer array 115 may be energized in a first imaging mode for imaging. The second transducer array 115 may be energized and acoustic energy 118 may be emitted from each of the transducers 114. The first transducer array 110 may detect or listen to the initial reflections or leading edge of the return signal 122 to image or evaluate the target region and/or tissue changes. It is contemplated the imaging process may last for approximately 10 microseconds. While the second transducer array 115 is described as emitting the acoustic energy and the first transducer array 110 as receiving the reflections, it is contemplated that the reverse configuration may also be used. For example, in some instances, the first transducer array 110 may emit acoustic energy while the second transducer array 115 receives the reflections. Further, while modulation system 100 is described as alternating between the first transducer array 110 and the second transducer array 115 as the imaging transducers, it is contemplated that the imaging may be performed by only one of the transducer arrays 110, 115.

Once the target region has been evaluated, both the first transducer array 110 and the second transducer array 115 may be energized in the second ablation mode, as shown in FIG. 7. Acoustic energy 116, 118 may be directed from both the first transducer array 110 and the second transducer array 115. In the ablation mode, acoustic energy 116, 118 may be directed from the transducers 112, 114 to form lesions in the desired target region. Acoustic energy 116, 118 may be directed towards the target region until another evaluation or image of the target region is desired. Energy delivery to the transducers 112, 114 may be stopped for a short ring-down period as shown in FIG. 8. The ring-down period may be a short time period relative the length of time the transducers 112, 114 are activated in the ablation mode. It is contemplated the ring-down period may be approximately 50 microseconds. The short duration of the imaging and the ring-down period may allow the transducers 112, 114 to be used almost all the time for ablation, thus maximizing the power delivery. After the ring-down period, the imaging and ablation cycles may be repeated. It is contemplated that the imaging and ablation cycles may be repeated as many times as necessary to achieve the desired tissue modulation. In some instances, the desired tissue modulation may be achieved after a single cycle, while in other instances, the desired tissue modulation may be require two, three, four, or more imaging and ablation cycles.

The modulation system 100 may be advanced through the vasculature in any manner known in the art. For example, system 100 may include a guidewire lumen to allow the system 100 to be advanced over a previously located guidewire. In some embodiments, the modulation system 100 may be advanced, or partially advanced, within a guide catheter such as the catheter 16 shown in FIG. 1. Once the transducers 112, 114 of the modulation system 100 have been placed adjacent to the desired treatment area, positioning mechanisms may be deployed, if so provided. While not explicitly shown, the transducers 112, 114 may be connected to a single control unit or to separate control units (such as control unit 18 in FIG. 1) by electrical conductors. As discussed above, the transducers 112, 114 may be connected to one or more control units, which may provide and/or monitor the system 100 with one or more parameters such as, but not limited to, frequency for performing the desired ablation procedure as well as imaging.

Once the modulation system 100 has been advanced to the treatment region, energy may be supplied to either the first transducer array 110 or the second transducer array 115 in a first imaging mode. Once the target region has been evaluated and/or imaged, energy may be supplied to both the first transducer array 110 and the second transducer array 115 in a second ablation mode. The amount of energy delivered to the transducer arrays 110, 115 may be determined by the desired treatment as well as the feedback provided by monitoring systems. After a predetermined time period or after predetermined treatment conditions have been met, the ablation may be stopped for a ring-down period. It is contemplated that the imaging, ablation, and ring-down period cycle may be repeated as many times as necessary to perform the desired tissue modulation.

In some instances, the elongate shaft 106 may be rotated and additional ablation can be performed at multiple locations around the circumference of the vessel 102. In some instances, a slow automated “rotisserie” rotation can be used to work around the circumference of the vessel 102, or a faster spinning can be used to simultaneously ablate around the entire circumference. The spinning can be accomplished with a distal micro-motor or by spinning a drive shaft from the proximal end. In other instances, the elongate shaft 106 may be indexed incrementally between desired orientations. In some embodiments, ultrasound sensor information can be used to selectively turn on and off the ablation transducers to warm any cool spots or accommodate for veins, or other tissue variations. The number of times the elongate shaft 106 is rotated at a given longitudinal location may be determined by the number and size of the transducer arrays 110, 115 on the elongate shaft 106. Once a particular location has been ablated, it may be desirable to perform further ablation procedures at different longitudinal locations. Once the elongate shaft 106 has been longitudinally repositioned, energy may once again be delivered to the transducer arrays 110, 115 to perform imaging and ablation as desired. If necessary, the elongate shaft 106 may be rotated to perform ablation around the circumference of the vessel 102 at each longitudinal location. This process may be repeated at any number of longitudinal locations desired. It is contemplated that in some embodiments, the system 100 may include transducer arrays 110, 115 at various positions along the length of the modulation system 100 such that a larger region may be treated without longitudinal displacement of the elongate shaft 106.

Those skilled in the art will recognize that the present invention may be manifested in a variety of forms other than the specific embodiments described and contemplated herein. Accordingly, departure in form and detail may be made without departing from the scope and spirit of the present disclosure as described in the appended claims.

What is claimed is:

1. An intravascular nerve modulation system comprising:
an elongate shaft having a proximal end region and a distal end region;
a first transducer array having one or more transducers disposed adjacent to the distal end region of the elongate shaft; and
a second transducer array having one or more transducers disposed adjacent to the distal end region of the elongate shaft;
wherein at least one of the first or second transducer arrays is configured to both send ultrasonic pulses and receive reflected pulses.

2. The intravascular nerve modulation system of claim 1, wherein the one or more transducers of the first transducer array and the one or more transducers of the second transducer array comprise dual-function transducers.

3. The intravascular nerve modulation system of claim 2, wherein the one or more transducers of the first transducer array and the one or more transducers of the second transducer array are tuned to a frequency of 20-40 megahertz.

4. The intravascular nerve modulation system of claim 2, wherein the one or more transducers of the first transducer array and the one or more transducers of the second transducer array are tuned to a frequency of approximately 5-10 megahertz.

5. The intravascular nerve modulation system of claim 1, further comprising a control unit.

6. The intravascular nerve modulation system of claim 5, wherein the control unit alternately supplies power to the first and second transducer arrays between a first imaging mode and a second ablation mode.

7. The intravascular nerve modulation system of claim 6, wherein in the first imaging mode only one of the first or second transducer arrays send ultrasonic pulses.

8. The intravascular nerve modulation system of claim 6, wherein in the second ablation mode both the first and second transducer arrays send ultrasonic pulses.

9. An intravascular nerve modulation system, comprising:
an elongate shaft having a distal end region;
a first transducer array including a plurality of transducers disposed along the distal end region;
a second transducer array including a plurality of transducers disposed along the distal end region;
wherein the first transducer array is configured to both send ultrasonic pulses and receive reflected pulses; and
wherein the second transducer array is configured to send ultrasonic pulses.

10. The intravascular nerve modulation system of claim 19, wherein the transducers of the first transducer array alternate with the transducers of the second transducer array along the distal end region of the elongate shaft.

11. A method for performing intravascular nerve modulation, the method comprising:
providing a nerve modulation system comprising:
an elongate shaft having a proximal end region and a distal end region;
a first transducer array having one or more transducers disposed adjacent to the distal end region of the elongate shaft; and
a second transducer array having one or more transducers disposed adjacent to the distal end region of the elongate shaft;
advancing the nerve modulation system through a lumen such that the distal end region of the elongate shaft is adjacent to a first target region;
supplying a first current to one of the first or second transducer arrays to generate a first acoustic energy;
receiving reflected pulses from the first acoustic energy at one of the first or second transducer arrays to image the first target region; and
supplying a second current to both the first and the second transducer arrays to generate a second acoustic energy different from the first acoustic energy.

12. The method of claim 11, further comprising a ring-down period.

13. The method of claim 11, wherein the first current is supplied to the first transducer array.

14. The method of claim 11, wherein the reflected pulses from the first acoustic energy are received by the second transducer array.

15. The method of claim 12, further comprising:
supplying a third current to one of the first or second transducer arrays to generate a third acoustic energy; and
receiving reflected pulses from the third acoustic energy at the first or second transducer array to image the first target region after the ring-down period.

16. The method of claim 15, further comprising supplying a fourth current to both the first and the second transducer arrays to generate a fourth acoustic energy different from the third acoustic energy.

17. The method of claim 15, wherein the third current is supplied to the second transducer array.

18. The method of claim 17, wherein the reflected pulses from the third acoustic energy are received by the first transducer array.

19. The method of claim 15, wherein the third current is supplied to the first transducer array.

20. The method of claim 19, wherein the reflected pulses from the third acoustic energy are received by the second transducer array.
